(19) United States
(12) Patent Application Publication
(10) Pub. No.: US 2010/0137700 A1
(43) Pub. Date: Jun. 3, 2010

(54) CATHETERS AND ELECTROPHYSIOLOGICAL DEVICES

(75) Inventors: Rod Passman, Chicago, IL (US); Jason Rubenstein, Brookfield, WI (US)

Correspondence Address: Casimir Jones, S.C., 2275 DEMING WAY, SUITE 310, MIDDLETON, WI, 53562, US

(73) Assignee: NORTHWESTERN UNIVERSITY, Evanston, IL (US)

(21) Appl. No.: 12/628,602

(22) Filed: Dec. 1, 2009

Related U.S. Application Data
(60) Provisional application No. 61/118,894, filed on Dec. 1, 2008.

(51) Int. Cl. A61B 5/042; A61B 6/00; A61M 25/00

(52) U.S. Cl. 600/374; 600/435

(57) ABSTRACT

The present invention provides systems and methods of use for catheters and electrophysiological devices. In particular, the present invention provides coronary catheters within which an extendable electrode array is housed.

The present Application claims priority to U.S. Provisional Application Ser. No. 61/118,894 filed Dec. 1, 2008, herein incorporated by reference in its entirety.

### FIELD OF THE INVENTION

The present invention provides systems and methods of use for catheters and electrophysiological devices. In particular, the present invention provides coronary catheters within which an extendable electrode array is housed.

### BACKGROUND OF THE INVENTION

Localization of coronary left lateral accessory pathways is currently commonly performed by placing a steerable decapolar electrophysiology catheter into the coronary sinus as laterally as possible. The pathway is located by identifying which electrode on the catheter has either fused atrial and ventricular signals or earliest signals during either pacing or sinus rhythm. The catheter ideally will “bracket” the pathway; that is to have electrodes septally and laterally to the pathway to confirm its position along the catheter. This maneuver is often limited by the lateral extent to which the catheter can be advanced; the coronary sinus becomes progressively smaller and tortuous laterally, so the catheter cannot be advanced beyond a certain point.

### SUMMARY

In some embodiments, the present invention provides a system comprising a decapolar coronary sinus catheter, wherein the catheter comprises an inner lumen, and an electrophysiological device, wherein the electrophysiological device is configured to fit within the inner lumen of said catheter. In some embodiments, the catheter comprises one or more distally located electrodes. In some embodiments, the catheter is steerable. In some embodiments, the catheter has an outer diameter of 3-8 Fr (French) (e.g., 3, 4, 5, 6, 7, 8). In some embodiments, the catheter is configured for injecting an imaging agent. In some embodiments, the electrophysiological device is extendable and/or retractable.

In some embodiments, the electrophysiological device comprises one or more distally located electrodes. In some embodiments, the electrophysiological device is steerable. In some embodiments, the electrophysiological device has an outer diameter ranging from about 0.5-4 Fr (e.g., 0.5, 1, 2, 3, 4).

In some embodiments, the present invention provides a kit comprising one or more decapolar coronary sinus catheters, wherein the catheters comprise an inner lumen, and one or more electrophysiological devices, wherein said electrophysiological devices are configured to fit within one or more of the inner lumens of the catheters. In some embodiments, the catheters and electrophysiological devices comprise a range of outer diameters. In some embodiments, the catheters and electrophysiological devices comprise a range of number of electrodes.

In some embodiments, the present invention provides a method of coronary localization comprising: placing a decapolar coronary sinus catheter into the lateral coronary sinus, wherein the catheter comprises an inner lumen, passing an electrophysiological device through the inner lumen of the catheter, extending the electrophysiological device deeper into the lateral coronary lumen, and localizing lateral coronary accessory pathways using electrodes located on the distal end of the electrophysiological device. In some embodiments, the present invention comprises an additional step, between placing the decapolar coronary sinus catheter and passing the wire through the inner lumen of the catheter, of localizing lateral coronary accessory pathways using electrodes located on the distal end of said catheter. In some embodiments, the present invention comprises an additional step, between placing the decapolar coronary sinus catheter and passing the electrophysiological device through the inner lumen of the catheter, of injecting an imaging agent through the inner lumen of the catheter into the lateral coronary sinus.

### BRIEF DESCRIPTION OF THE DRAWINGS

The foregoing summary and detailed description is better understood when read in conjunction with the accompanying drawings which are included by way of example and not by way of limitation.

FIG. 1 shows a schematic of a side view of an exemplary steerable coronary sinus catheter with the inner electrophysiological device retracted.

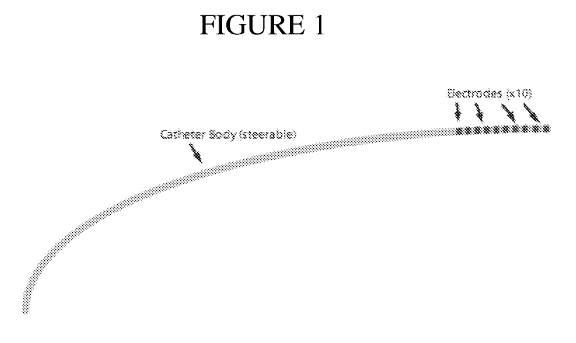

FIG. 2 shows a schematic of a side view of an exemplary steerable coronary sinus catheter with the inner electrophysiological device extended.

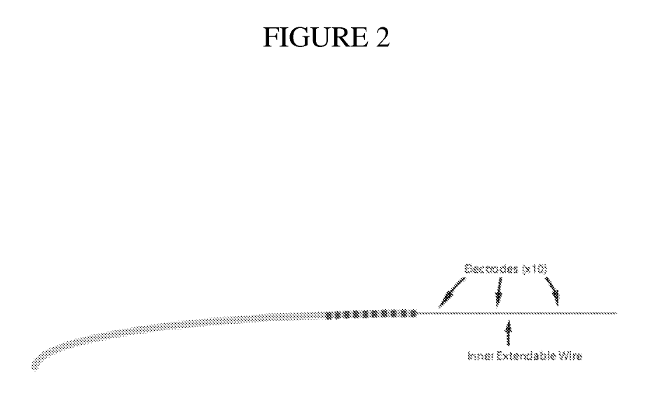

### DETAILED DESCRIPTION OF EMBODIMENTS

In some embodiments, the present invention provides systems, devices, kits, and methods for accessing pathways within a subject (e.g. that are beyond the reach of standard catheters). In some embodiments, the present invention provides accessing blood vessel pathways (e.g. coronary pathways (e.g. left lateral coronary accessory pathways)). In some embodiments, the present invention provides localization of left lateral coronary accessory pathways, for example, those beyond the reach of standard sized catheters. In some embodiments, the pathway is identified by determining which electrode on an electrophysiological device has either fused atrial and ventricular signals or earliest signals during either pacing or sinus rhythm. In some embodiments, the electrophysiological device “brackets” the pathway (e.g. electrodes placed septally and laterally to the pathway to confirm its position along the electrophysiological device). In some embodiments, the present invention provides a steerable decapolar coronary sinus catheter with an inner lumen capable of housing an extendable electrode array extendable electrode array. In some embodiments, the electrophysiological device extends beyond the reach of the catheter, due to the smaller diameter of the electrophysiological device. In some embodiments, the electrophysiological device is configured to extend in to regions and pathways not accessible to the catheter. In some embodiments, the electrophysiological device is configured for electrophysiology mapping of regions and pathways not accessible to standard catheters. It should be understood that the catheters of the invention may find use in a wide variety of settings, and are not limited to operation in the coronary sinus, or to electrophysiological mapping.

In some embodiments, the present invention provides a method of coronary pathway localization. It is known to one in the art, that coronary pathways can be identified by extending an electrophysiological catheter into the coronary sinus, and using electrodes to “bracket” the pathway; that is to have electrodes septally and laterally to the pathway to confirm the pathway's position along the catheter. This maneuver is often limited by the lateral extent to which the catheter can be advanced, as the coronary sinus becomes progressively smaller and tortuous laterally, so the catheter cannot be advanced beyond a certain point. In some embodiments, the present invention provides methods to allow mapping and localization beyond the capabilities of standard catheters. In some embodiments of the present invention, a decapolar coronary sinus catheter is placed into the lateral coronary sinus. In some embodiments, the catheter is placed as far, or nearly as far, as its size (e.g. diameter, or circumference) will permit. In some embodiments, upon placement of the catheter, an electrophysiological device is passed through the inner lumen of the catheter, extending the electrophysiological device deeper into the lateral coronary lumen. In some embodiments, the electrophysiological device is configured for extension further into the coronary sinus than the catheter (e.g. the electrophysiological device has a smaller diameter than the catheter). In some embodiments, the electrophysiological device is configured for localizing lateral coronary accessory pathways using electrodes located on the distal end of the electrophysiological device. In some embodiments, the present invention provides a method for mapping coronary pathways beyond the reach of a catheter. The present invention is not limited to use in the coronary sinus. In some embodiments, the present invention finds use for mapping and localization in environments in which decreased size and increased mobility are desired. The present invention is not limited to mapping and localization functionalities.

In some embodiments, the present invention finds use in delivery of therapeutics, ablation therapy, or other functions in which increased access and mobility are desired.

In some embodiments, devices of the present invention are inserted blood vessels to provide access to regions of interest within a subject. In some embodiments, catheter devices are inserted into, deployed through, and/or targeted to arteries or veins including, for example, the ascending aorta, right coronary artery, left coronary artery, anterior interventricular, circumflex, left marginal arteries, posterolateral artery, intermedius, arch of aorta, brachiocephalic artery, common carotid artery, internal carotid artery, external carotid artery, subclavian artery, vertebral artery, internal thoracic artery, thyrocervical trunk, deep cervical artery, dorsal scapular artery, brachial artery, thoracic aorta, abdominal aorta, inferior phrenic, celiac, superior mesenteric, middle suprarenal, renal, anterior and posterior, interlobar artery, gonadal, lumbar, inferior mesenteric, median sacral, common iliac, common iliac arteries, internal iliac artery, anterior division, obturator artery, superior vesical artery, vaginal artery (females), inferior vesical artery (males), middle rectal artery, internal pudendal artery, inferior gluteal artery, uterine artery (females), deferential artery (males), (obliterated) umbilical artery, posterior division, iliolumbar artery, lateral sacral artery, superior gluteal artery, external iliac artery, inferior epigastric artery, deep circumflex iliac artery, femoral artery, superficial epigastric artery, superficial circumflex iliac artery, superficial external pudendal artery, deep external pudendal artery, deep femoral artery, descending genicular artery, popliteal artery, anterior tibial artery, posterior tibial artery, sural artery, medial superior genicular artery, lateral superior genicular artery, middle genicular artery, inferior lateral, and inferior medial genicular artery.

In some embodiments, the present invention may be used with any subject or patient, including, but not limited to, humans, non-human primates, mammals, feline, canine, bovine, equine, porcine, rodent, etc. In some embodiments, the subject is a human requiring a medical procedure (e.g. surgery). In some embodiments, the subject is a human or other mammal suffering from a condition, disease, or disorder in which electrophysiology mapping is useful and/or required with treatment. In some embodiments, the subject is a human or other mammal undergoing surgery or catheter based diagnostic or therapeutic procedures. In addition, any body region may be used with the devices, systems, kits, and methods of the present invention.

In some embodiments, the present invention comprises a catheter (e.g. a decapolar coronary sinus catheter). In some embodiments, the catheter shaft is flexible (e.g., bendable). In some embodiments the catheter is flexible throughout its length. In some embodiments the catheter is flexible at its distal end. In some embodiments, the catheter is substantially non-compressible along its length. In some embodiments, the catheter comprises flexible regions, and more rigid regions. The catheter shaft can be of any suitable construction and made of any suitable material. A suitable construction includes, but is not limited to, an outer wall made of polyurethane, TEFLON, HDPE, nylon, PEEK, PTFE, PEBAX, or other suitable materials. In some embodiments, devices comprise materials such as CoCrMo alloy, Titanium alloy, cpTi, Ti6A14V ELI medical grade stainless steel, Tantalum, Tantalum alloy, Nitinol, polymers, alloys, metals, ceramics, oxides, minerals, glasses and combinations thereof. In some embodiments, a device of the present invention is made of any suitable material or materials for the production of medical devices. In some embodiments, materials are selected based on the specific application, and/or deployment location. In some embodiments, a device of the present invention comprises one or more metals, alloys, plastics, polymers, natural materials, synthetic materials, fabrics, etc. In some embodiments, a device of the present invention comprises one or more metals including but not limited to aluminum, antimony, boron, cadmium, cesium, chromium, cobalt, copper, gold, iron, lead, lithium, manganese, mercury, molybdenum, nickel, platinum, palladium, rhodium, silver, tin, titanium, tungsten, vanadium, and zinc. In some embodiments, a device of the present invention comprises one or more alloys including but not limited to alloys of aluminium (e.g., Al—Li, alumel, duralumin, magnox, zamak, etc.), alloys of iron (e.g., steel, stainless steel, surgical stainless steel, silicon steel, tool steel, cast iron, Spiegeleisen, etc.), alloys of cobalt (e.g., stellite, talonite, etc.), alloys of nickel (e.g., German silver, chromel, mumetal, monel metal, nichrome, nicrosil, nisil, nitinol, etc.), alloys of copper (beryllium copper, billon, brass, bronze, phosphor bronze, constantan, cupronickel, bell metal, Devarda's alloy, gilding metal, nickel silver, nordic gold, prince's metal, tumbaga, etc.), alloys of silver (e.g., sterling silver, etc.), alloys of tin (e.g., Britannium, pewter, solder, etc.), alloys of gold (electrum, white gold, etc.), amalgam, and alloys of lead (e.g., solder, terne, type meta, etc.). In some embodiments, a device of the present invention comprises one or more plastics including but not limited to Bakelite, neoprene, nylon, PVC, polystyrene, polyacrylonitrile, PVB, silicone, rubber, polyamide, synthetic rubber, vulcanized rubber, acrylic, polyethylene, polypropylene, polyethylene terephthalate, polytetrafluoroethylene, gore-tex, polycarbonate, etc. In some embodiments, elements of a device of the present invention a device of the present invention may also comprise glass, textiles (e.g., from animal, plant, mineral, and/or synthetic sources), liquids, etc.

In some embodiments, the catheter outer wall comprises an imbedded braided mesh of stainless steel or the like, as is generally known in the art, to increase torsional stiffness of the catheter shaft so that, when the proximal catheter handle is rotated, the distal catheter shaft will rotate in a corresponding manner. In some embodiments, torsional stiffness is achieved through other mechanisms known to those in the art. In some embodiments, the useful length of the catheter, e.g., that portion that can be inserted into the body can vary as desired. In some embodiments, the useful length ranges from about 30 cm to about 250 cm (e.g. 30 cm . . . 50 cm . . . 100 cm . . . 150 cm . . . 200 cm . . . 250 cm, and lengths therein). In some embodiments, the diameter, circumference, and/or gauge of the catheter can vary as desired. In some embodiments, useful outer diameters range from about 3-8 French (Fr) (e.g. 3 Fr . . . 4 Fr . . . 5 Fr . . . 6 Fr . . . 7 Fr . . . 8 Fr, and diameters therein). In some embodiments, the catheter is steerable to allow for navigation within a subject or working environment (e.g. coronary sinus, blood vessels, body cavity, etc.). In some embodiments, the catheter has bidirectional steerablity (e.g.

the distal end of the catheter is configured to be bendable in the left/right plane via controls at the sheath handle), and/or rotational steerability (e.g. the distal end of the catheter is configured to have 360° bendability). One suitable steerable catheter is described in U.S. Pat. No. 5,656,029, herein incorporated by reference in its entirety.

In some embodiments, the present invention provides a catheter comprising an inner lumen. In some embodiments, the lumen is configured to provide an environment for housing and/or delivering compositions (e.g. an imaging agent), systems, or devices (e.g. an electrophysiological device, electrode array, etc.) to desired locations (e.g. blood vessels, lateral coronary sinus, etc.). In some embodiments, the lumen of the catheter may find utility in navigation, localization, mapping, or therapeutics within a desired work space (e.g. within a subject, animal, mammal, human, tissue, organ, the coronary sinus, etc.). In some embodiments, the inner lumen is configured for injection of a composition (e.g. imaging agent (e.g. contrast agent)) and/or depositing a composition at the distal end of the catheter. In some embodiments, the lumen is a delivery chamber, configured to deliver an agent or device to the distal end of the catheter. In some embodiments, the lumen is configured to deliver a liquid and/or solid composition. In some embodiments, the lumen is configured to house an electrophysiological device, which is capable of extending and retracting from the distal end of the lumen. In some embodiments, the lumen is configured for multiple functions (e.g. housing an extendable electrode array and injection of a composition). In some embodiments, the inner diameter of the lumen may vary as desired. In some embodiments, the lumen has an inner diameter of 1-7 French (e.g., 1, 2, 3, 4, 5, 6, 7). In some embodiments, the diameter of the lumen is directionally proportional to the diameter of the catheter. In some embodiments, the diameter of the lumen is directly proportional to the diameter of the electrophysiological device it houses. In some embodiments, the diameter of the lumen is based on its specific set of functions (e.g. injection of imaging agent, extending electrophysiological mapping probe, etc.).

In some embodiments, the distal end of the catheter comprises a mapping assembly. In some embodiments the mapping assembly comprises one or more electrodes at the distal end of the catheter (e.g. a series of electrodes evenly spaced across the end of the catheter). In some embodiments, the mapping assembly provides localization information. In some embodiments, the mapping assembly is formed of a non-conductive cover, of any cross-sectional shape as desired (e.g. a generally tubular cover). The non-conductive cover can be pre-formed into the desired generally shape. In some embodiments, the mapping assembly comprises electrodes configured to perform mapping, localization, and navigation. The number of electrodes on the assembly can vary as desired. In some embodiments, the number of electrodes ranges from about two to about twenty (e.g. 2 electrodes, 3 electrodes, 4 electrodes, 5 electrodes, 6 electrodes, 7 electrodes, 8 electrodes, 9 electrodes, 10 electrodes, 11 electrodes, 12 electrodes, 13 electrodes, 14 electrodes, 15 electrodes, 16 electrodes, 17 electrodes, 18 electrodes, 19 electrodes, or 20 electrodes). In some embodiments, the number of electrodes ranges from about eight to about twelve. In some embodiments, the electrodes are approximately evenly spaced. In some embodiments, a distance of approximately 1-20 mm is provided between the centers of the electrodes. In some embodiments, wires attached to the electrodes extend through the lumen of the distal shaft, through the catheter body, and terminate at the proximal end of the catheter. In some embodiments, the portion of the lead wires extending through the central lumen of the catheter is enclosed within a protective sheath, which can be made of any suitable material. The device is not limited to particular types or kinds of electrodes. Indeed, any type of electrode approved for clinical use may be used with the devices of the present invention. In some embodiments, the same type of electrodes are positioned along the device. In some embodiments, two or more different types of electrodes are positioned along the device. The devices are not limited to a particular positioning of the electrodes along the device. In some embodiments, the electrodes are symmetrically positioned along the device. In some embodiments, the electrodes are randomly positioned along the device. In some embodiments, the positioning of the electrodes along the device renders the device capable of mapping measurements. The electrodes positioned along a device are not limited to a particular function. In some embodiments, one or more of the electrodes are configured to emit or apply current (e.g. direct or alternating) or voltage. In some embodiments, one or more of the electrodes are configured to measure voltage in the surrounding area. In some embodiments, one or more of the electrodes are configured to emit or apply a current and/or to measure voltage in the surrounding region. In some embodiments, electrodes are not limited to emit or apply a particular amount of current. In some embodiments, the current applied or emitted by electrodes is not greater than 100 milliamperes (mA) (e.g., <100 mA, <90 mA, <80 mA, <70 mA, <60 mA, <50 mA, <40 mA, <30 mA, <20 mA, <10 mA, <9 mA, <8 mA, <7 mA, <6 mA, <5 mA, <4 mA, <3 mA, <2 mA, <1 mA, etc.). In some embodiments, the current applied or emitted by electrodes positioned along the device is greater than 0.01 mA (e.g., >0.01 mA, >0.05 mA, >0.1 mA, >0.2 mA, >0.3 mA, >0.4 mA, >0.5 mA, >0.6 mA, >0.6 mA, >0.8 mA, >0.9 mA, >1 mA, >2 mA, >3 mA, >4 mA, >5 mA, >6 mA, >7 mA, >8 mA, >9 mA, >10 mA, etc.). In some embodiments, the current applied or emitted by electrodes positioned along the device is between 0.1 and 10 mA (e.g., >0.1 and <2 mA, >0.2and <1.5 mA, >0.5 and <1.3 mA, etc.). In some embodiments, the present invention provides one or more sensing electrodes, or electrodes configured to sense currents or voltages in the local region in which they are deployed. In some embodiments, the electrodes detect currents emitted by other electrodes. In some embodiments, sensing electrodes detect physiological currents or voltages.

In some embodiments, the present invention provides an electrophysiological device which is configured to be deployed from within the lumen of a catheter. In some embodiments, the electrophysiological device is an extendable wire located within the lumen of a catheter. In some embodiments, the electrophysiological device comprises one or more electrodes at the distal end of the wire (e.g. a series of electrodes evenly spaced across the end of the wire). In some embodiments, the electrophysiological device provides localization of pathways within a subject, organ, or tissue (e.g. lateral coronary accessory pathways by bracketing the pathway using electrodes along the extended electrode array). In some embodiments, the electrophysiological device provides localization of left lateral coronary accessory pathways beyond the reach of a standard diameter catheter (e.g. 6 French). In some embodiments, the electrophysiological device is a wire used to detect electrical activity. In some embodiments, the wire is deployed into the coronary sinus from within the lumen of a coronary sinus catheter. In some embodiments, the electrophysiological device is configured to be housed within the lumen of a coronary sinus catheter. In some embodiments, the electrophysiological device is formed of a non-conductive cover, of any cross-sectional shape as desired (e.g. a generally tubular cover). The non-conductive cover can be pre-formed into the desired generally shape. In some embodiments, the electrophysiological device comprises electrodes configured to perform mapping, localization, and navigation. In some embodiments, the electrophysiological device comprises an electrode array and/or electrophysiology mapping device. The number of electrodes on the assembly can vary as desired. In some embodiments, the electrophysiological device comprises one or more distally located electrodes (e.g. 2 electrodes, 3 electrodes, 4 electrodes, 5 electrodes, 6 electrodes, 7 electrodes, 8 electrodes, 9 electrodes, 10 electrodes, 11 electrodes, 12 electrodes, 13 electrodes, 14 electrodes, 15 electrodes, 16 electrodes, 17 electrodes, 18 electrodes, 19 electrodes, or 20 electrodes). In some embodiments, the electrodes are approximately evenly spaced. In some embodiments, a distance of approximately 1-20 mm is provided between the centers of the electrodes. In some embodiments, distally located electrodes may be used to detect electrical activity in a subject or tissue. In some embodiments, the electrophysiological device has a plurality of sensing electrodes, disposed on the distal portion for detection of electrical activity. In some embodiments, electrodes are position on the surface of a wire. In some embodiments, the electrophysiological device comprises a wire, housed within insulation, wherein a plurality of windows in the insulation creates discrete sensible/detectable positions along the length of the wire. In some embodiments, the insulation can be made of any suitable material. As with the catheter electrodes above, the electrodes of the extendable electrophysiology device comprise any number of suitable configuration for mapping, navigation, and/or localizing within a desired space (e.g. blood vessel, coronary pathways, left coronary sinus, etc.).

In some embodiments, control of the catheter is provided by an integrated hand-held control mechanism and/or handle mounted on the proximal end of the catheter. In some embodiments, control of the catheter is provided by an external control unit. In some embodiments, the control mechanism/handle can be of various types, and adapted for operating a steerable catheter wherein the bend of the catheter can be selectively controlled by the operator. In some embodiments, the mechanism/handle includes a set of control switches, which allow the operator to control the steering of the catheter and other operational functions of the catheter. It will be apparent to one of ordinary skill in the art that other control mechanisms/handles can be employed with the systems of the invention without departing from the scope thereof. Specifically, other systems can include joystick controls for operating the steerable catheters and can include controls for rotating the angle at which the distal end of the catheter bends. Other modifications and additions can be made to the control mechanism/handle without departing from the scope of the invention. In some embodiments, the control mechanism/handle also controls extension of an inner device (e.g. electrode, electrode array, etc.) from the catheter, steering of any devices housed within the catheter, injection/ejection of any agents from the catheter, or any other functions that are understood by one in the art.

In some embodiments, the present invention comprises one or more processors, microprocessors, or controllers. Devices are not limited to particular types of processors, microprocessors, or controllers. The devices are not limited to particular uses for the processors. In some embodiments, the devices utilize processors that monitor and/or control and/or provide feedback concerning steering, localization, measurements, and/or mapping. In some embodiments, a processor directs some or all of the measurement functions of a device. In some embodiments, a processor is involved in all or some of electrode operation, protocol selection, data storage, data analysis, memory access, data display, etc. In some embodiments, the processor creates electrophysiology maps through comparing the measured currents. In some embodiments, measured currents are compared with each other and/or with standardized information (e.g., standardized currents).

In some embodiments, a processor, microprocessor, or controller is provided within a computer module or central processing unit (CPU). The computer module may also comprise software that is used by the processor to carry out one or more of its functions. For example, in some embodiments, the present invention provides software for regulating the frequency of how currents and/or voltages are measured, different measurement protocols, analysis protocols, etc. In some embodiments, the software is configured to provide information (e.g., monitoring information) in real time. In some embodiments, the processor is configured for the creation of a database of information.

In some embodiments, the devices are configured to present and/or display information. The devices are not limited to a particular manner of presenting and/or displaying information. In some embodiments, the devices have therein a display region configured to display information (e.g. on the handle portion of a catheter device). The devices are not limited to displaying particular information. In some embodiments, the information is the actual measured currents or voltages. In some embodiments, an electrophysiology map is displayed. Catheter devices are not limited to a particular manner of displaying information within the display region. In some embodiments, the information is displayed audibly (e.g., various sounds (e.g., alarms) specific for types of information). In some embodiments, the information is displayed visually (e.g., lights, display screen, or printer component). In some embodiments, a device provides a LCD screen for displaying device information (e.g., power, current settings, current and/or voltage measurements, measurement history, activity, etc.). In some embodiments, the present invention provides a memory element configured to store data. Catheter devices are not limited to a particular type or kind or storage capacity for the memory element. In some embodiments, the present invention provides a processor which operates in conjunction and in communication with the memory element.

Catheter devices of the present invention are not limited to a particular type or kind of energy consumption. In some embodiments, a device of the present invention provides an energy module (e.g., battery module, AC power module, DC power module, etc.). In some embodiments, the device provides an element for AC power from an outlet (e.g., 120V/220V outlet). In some embodiments, a device provides a battery module. In some embodiments, a battery module provides sufficient power to a device of the present invention. In some embodiments, a battery module provides sufficient energy to power all of the functionalities of a device of the present invention.

In some embodiments, the present invention provides kits comprising one or more decapolar coronary sinus catheters, wherein the catheters comprise an inner lumen, and one or more electrophysiological devices, wherein said electrophysiological devices are configured to fit within one or more of the inner lumens of the catheters. In some embodiments, it is desirable to maximize access to small environments, and thus a smaller diameter electrophysiological device finds use. In some embodiments, a larger number of electrodes are desired, and thus a larger electrophysiological device finds use. In some embodiments, additional features on the electrophysiological device are desired, and thus a larger electrophysiological device finds use. In some embodiments, the present invention provides kits providing two of more electrophysiological devices and catheters of varying sizes (e.g. diameter, length, etc.) and functionalities (e.g. different number of electrodes, materials, organizations, steerablity, etc). In some embodiments, a kit may further comprise written instructions, software, or other materials useful in operation the catheter and electrophysiological devices.

We claim:

1. A system comprising a decapolar coronary sinus catheter, wherein said catheter comprises an inner lumen; and an electrophysiological device, wherein said electrophysiological device is configured to fit within said inner lumen of said catheter.

2. The system of claim 1, wherein said catheter comprises one or more distally located electrodes.

3. The system of claim 1, wherein said catheter is steerable.

4. The system of claim 1, wherein said catheter has an outer diameter of 3-8 Fr.

5. The system of claim 1, wherein said catheter is configured for injecting an imaging agent.

6. The system of claim 1, wherein said electrophysiological device is extendable and retractable from within said catheter.

7. The system of claim 1, wherein said electrophysiological device comprises one or more distally located electrodes.

8. The system of claim 1, wherein said electrophysiological device is steerable.

9. The system of claim 1, wherein said electrophysiological device has an outer diameter of 0.5-4 Fr.

10. A kit comprising one or more decapolar coronary sinus catheters, wherein said catheters comprise an inner lumen; and one or more electrophysiological devices, wherein said electrophysiological devices are configured to fit within one or more of said inner lumens of said catheters.

11. The kit of claim 10, wherein said catheters and said electrophysiological devices comprise a range of outer diameters.

12. The kit of claim 10, wherein said catheters and said electrophysiological devices comprise a range of number of electrodes.

13. A method of coronary localization comprising
a) placing a decapolar coronary sinus catheter into the lateral coronary sinus, wherein said catheter comprises an inner lumen;
b) passing an electrophysiological device through said inner lumen of said catheter;
c) extending said electrophysiological device deeper into the lateral coronary lumen; and
d) localizing lateral coronary accessory pathways using electrodes located on the distal end of said electrophysiological device.

14. The method of claim 13, further comprising an additional step between steps (a) and (b) comprising localizing lateral coronary accessory pathways using electrodes located on the distal end of said catheter.

15. The method of claim 13, further comprising an additional step between steps (a) and (b) comprising injecting an imaging agent through said inner lumen of said catheter into said lateral coronary sinus.
